(19) United States
(12) Patent Application Publication
(10) Pub. No.: US 2015/0250421 A1
(43) Pub. Date: Sep. 10, 2015

(54) CONDUCTIVE NANOCRYSTALLINE DIAMOND MICRO-ELECTRODE SENSORS AND ARRAYS FOR IN-VIVO CHEMICAL SENSING OF NEUROTRANSMITTERS AND NEUROACTIVE SUBSTANCES AND METHOD OF FABRICATION THEREOF

(71) Applicant: ADVANCED DIAMOND TECHNOLOGIES, INC., Romeoville,, IL (US)

(72) Inventors: Prabhu U. Arumugam, Ruston, LA (US); Shabnam Siddiqui, Ruston, LA (US); Hongjun Zeng, Naperville, IL (US)

(21) Appl. No.: 14/431,653

(22) PCT Filed: Sep. 26, 2013
(86) PCT No.: PCT/US13/61958

§ 371 Date: Mar. 26, 2015

Related U.S. Application Data
(60) Provisional application No. 61/705,715, filed on Sep. 26, 2012.

(51) Int. Cl. A61B 5/00; A61B 5/145; A61B 5/1486

(52) U.S. Cl. CPC A61B 5/685; A61B 5/14865; A61B 5/14546; A61B 5/0048; A61B 2562/125

(57) ABSTRACT

Conductive diamond micro-electrode sensors and sensor arrays are disclosed for in vivo chemical sensing. Also provided is a method of fabrication of individual sensors and sensor arrays. Reliable, sensitive and selective chemical micro-sensors may be constructed for real-time, continuous monitoring of neurotransmitters and neuro-active substances in vivo. Each sensor comprises a conductive microwire, having a distal end comprising a tip, coated with nanocrystalline or ultrananocrystalline conductive diamond, and an overlying insulating layer. Active sensor areas of the conductive diamond layer are defined by openings in the insulating layer at the distal end. Multiple sensor areas may be defined by a 2 or 3 dimensional pattern of openings near the tip. This structure limits interference from surrounding areas for improved signal to noise ratio, sensitivity and selectivity. Using fast-scan cyclic voltammetry and high speed multiplexers, multiple sensors can be arrayed to provide 3-D spatial, and near real-time monitoring.

### CROSS-REFERENCE TO RELATED APPLICATION

This application claims priority from U.S. provisional patent application Ser. No. 61/705,715 entitled “UNCD Microsensors for In Vivo Monitoring of Neurotransmitters”, filed Sep. 26, 2012, which is incorporated herein by reference, in its entirety.

### TECHNICAL FIELD

This invention relates to sensors for the detection of neurotransmitters and neuroactive substances. More specifically, this disclosure relates to in vivo chemical micro-sensors for selective neurotransmitter sensing with real-time, continuous monitoring.

### BACKGROUND ART

Neurosensing has been in development for many years to monitor and detect changes and functions of the nervous system, including the brain. As the understanding of the nervous system has grown, so has the research and development of methods to detect and process chemicals related to how the nervous systems works and responds to different stimuli such as assorted biological materials and chemicals.

Sensors can be implanted to detect chemicals on the surface of the brain and monitor these levels to determine if intervention is indicated for the wellness of the patient. Sensors have also been used to detect environmental stimulation and then to stimulate other organs to produce a reaction. For example, these technologies have been used to detect light and then stimulate specific nerves or areas of the brain to provide a response analogous to natural visual processing.

Neurotransmitters can be monitored to determine the state of the systems and through this, preventative action can be taken. Detection of neurotransmitters in the brain and how the brain responds to them is being used as a research tool, for example to monitor assorted chronic diseases, disorders, and injuries. Furthermore, detection of neurotransmitters can potentially help to predict impending disorders so that some form of remedial action can take place to enhance the wellbeing of the patient.

Preferably, sensors need to be placed in the areas that the activity is most likely to be initiated. These locations could be deep within an organ, such as the brain.

To sense the neurotransmitter chemicals in these locations, microelectrodes have been developed. Some are provided as an array to be able to sense over a larger area while keeping the total contact area to a minimum. Some of these electrode devices are relatively long compared to the diameter so that they may reach deeper into the organ.

There are many different chemical neurotransmitters/neurochemicals used in brain function and throughout the body. One of the most common is dopamine which is used in many systems including the nervous, cardiovascular, hormonal and renal systems.

Another neurotransmitter that has been studied often is adenosine which is also important in cardiovascular and nervous systems. There are many other neurotransmitters that come in the form of amino acids, mono-amines, and peptides, for example.

Various electro-analytical techniques may be used to determine neurotransmitter activity at a micro-electrode probe tip. A recent key advance has been the development of fast-scan cyclic voltammetry (FSCV), which affords a superior combination of temporal and chemical resolution compared to other electro-analytical techniques. Sufficiently rapid rates of detection have been achieved for measurements of single cells to capture ultrafast single exo-cytotic release events.

Chemical resolution for FSCV is provided by a voltammogram, which is collected at each time point and functions as a chemical signature to identify the measured chemical species. FSCV has more recently been improved by the use of principal component regression, which is a chemometrics approach that permits statistical resolution of individual analytes from a mixed-analyte signal. FSCV has the capability of measuring major neuroactive substances in the brain, including dopamine, serotonin, norepinephrine, epinephrine, adenosine, histamine, nitric oxide, oxygen, and hydrogen peroxide. As an example, the utility of FSCV for investigating brain-behavior relationships has been demonstrated by studies monitoring dopamine in real time in ambulatory animals during goal-directed behavior and administration of abused drugs. The particular combination of temporal and chemical resolution makes FSCV a logical choice for real time monitoring of neuroactive substances.

Carbon-fiber microelectrodes (CFM) are currently considered to be the state-of-the-art microsensor for FSCV. These microelectrodes comprise a small diameter carbon-fiber, which is protected within an insulating layer, such as glass. The probe tip of carbon fiber is exposed and may be sharpened. A connection for support and electrical connection is provided at the other end. The small size (˜5- to 10 μm diameter) of the CFM reduces tissue damage, especially compared to a conventional microdialysis probe (˜300 μm diameter). The latter tends to compromise capillary blood flow in the sample region, disrupt neurotransmission, and induce neuronal trauma. A CFM affords a spatial resolution in the micron range and, when combined with extended-scan FSCV, provides a detection limit of ˜15 nM in the brain. However, the increased sensitivity of extended-scan FSCV is a trade-off against reduced response time of the CFM.

In the process of using a microelectrode, because of its small size, it can quickly lose its electro-activity induced by chemical reactions on the electrode such as oxidation. A microelectrode or probe can become “fouled” through damaged organic material blocking the sensing of the analyte neurotransmitter materials.

For FSCV measurements in freely behaving animals, the typical approach is to lower a fresh CFM acutely via a detachable microdrive. Use of a fresh CFM overcomes the diminution of response characteristics observed with long term implantation. Replacing the insulating layer of borosilicate glass with polyimide-coated fused silica extends the lifetime the CFM for longer term measurements. However, the ability to perform repeated measurements over weeks to months remains a highly prized, but as yet unrealized, goal of neurotransmitter monitoring. It is technically challenging and not commonplace with this configuration of CFM.

An additional limitation is that available CFMs are currently offered only as a single-probe microsensor. Microelectrode Arrays (MEAs) for electroanalytical techniques for sensing of neuroactive substances are known, and these have been fabricated using platinum on ceramic or silicon substrates or using pyrolyzed photoresist film on a silicon substrate. The latter provides a surface similar to carbon fiber. Use of MEAs provides concurrent, multi-site recording of brain activity. However, the size of the neurotransmitter probes developed to date limits their utility. Also, the necessary hardware and software supporting simultaneous measurements using greater than 4 channels has not yet been realized.

Emerging carbon nanomaterials such as nanotubes, nanofibers and micro-nanocrystalline diamond, have spurred renewed interest in investigating electrode material technology that is highly biocompatible, highly resistive to surface fouling and functional for extended time periods in chronic implants. Carbon nanotubes have been used on the electrode surface to provide a relevantly inert surface area in contact with biomaterials. Carbon is a good conductor and relevantly strong. Among carbon nanomaterials, conductive boron-doped diamond (BDD), in particular, offers excellent chemical, electrochemical and bio stability. BDD is an excellent electrode material for in vivo neurotransmitter analysis due to its intrinsic properties such as a wide electrochemical potential window, very small background “charging” current, chemical inertness and dimensional stability, increased working lifetime due to excellent biocompatibility, improved specificity, mechanical durability, pH-independent low background current, and high sensitivity due to weak adsorption of biomolecules such as proteins and oxidized products.

BDD is broadly classified into three categories based on its crystallite size: microcrystalline (MCD), nanocrystalline (NCD) and ultrananocrystalline (UNCD). NCD and MCD surfaces are sufficiently rough (Rₐ of ˜10-100 nm and 500-1000 nm RMS, respectively) to increase the risk of tissue damage. For implantable electrodes, UNCD with its as-deposited near atomic-scale smoothness (e.g., Rₐ of ˜5-8 nm RMS) offers an excellent choice.

Currently, diamond microelectrodes are custom-fabricated in several academic laboratories. In spite of many advantages, the diamond deposition on metal microwires requires careful surface preparation to seed the surface selectively with diamond nanoparticles, and achieving films that are continuous and pin-hole free is challenging due to the low re-nucleation rate associated with diamond growth chemistries. The commercial fabrication of diamond-based MEA's is also greatly hindered by the general incompatibility of diamond with wafer-scale microfabrication technologies for various reasons, including the large mismatch between the thermal expansion coefficient of diamond and typical semiconductor substrates and difficulties in planarization of hard and rough MCD layers. For NCD there can be challenges in controlling crystal size and high sp² content in grain boundaries results in heterogeneous film properties. Currently, it is impractical for laboratories to fabricate high-quality diamond microsensors at a reasonable cost.

The following references provide some examples of the fabrication and use of microelectrode sensors in-vivo chemical sensing of neurotransmitters and neuroactive substances:
Broderick et al., P. A., Identification, diagnosis, and treatment of neuropathologies, neurotoxicities, tumors, and brain and spinal cord injuries using microelectrodes with microvoltammetry. U.S. Pat. No. 7,112,319, 26 Sep. 2006.
Mech et al., B. V., Implantable device using ultra-nanocrystalline diamond. U.S. Pat. No. 7,127,286, 24 Oct. 2006.
Brabec et al., S. J., Medical devices incorporating carbon nanotube material and methods of fabricating same. U.S. Pat. No. 7,844,347, 30 Nov. 2010
Greenberg et al., R. J., Implantable Device for the Brain. US Patent Publication Number US 2009/0124965, 14 May 2009.
Scarsbrook, G. A., High Uniformity Boron Doped Diamond Material. Publication Number US 2010/0012491
Marinesco et al., S., Microsensor for Detection of D-Amino-Acids. Publication Number US 2010/0163432.
Qiang et al., L., Sensors for Analyte Detection and Methods of Manufacture Thereof. Publication Number US 2011/0315563.
Feldman et al., B. J., Analyte Sensors, Including, Nanomaterials and Methods of using the same. Publication Number US 2011/0046466.
Huffman, M. L and Venton, B. J. Electrochemical Properties of Different Carbon-Fiber Microelectrodes Using Fast-Scan Cyclic Voltammetry. Electroanalysis 20, 2008, No. 22, 2422-2428
Roy, P., et al., Selective Detection of Dopamine and Its Metabolite, DOPAC, in the Presence of Ascorbic Acid Using Diamond Electrode Modified by the Polymer Film, Electroanalysis, 2004, Vol. 16, Issue 21, p. 1777-1784.
Suzuki, A., Ivandini, T. A., Yoshimi, K., Fujishima, A., Oyama, G., Nakazato, T., Hattori, N., Kitazawa, S & Einaga, Y., Fabrication, Characterization, and Application of Boron-Doped Diamond Microelectrodes for in Vivo Dopamine Detection, Anal. Chem., 2007, 79, p. 8608-8615.
Swamy, K., Venton, B. J., Subsecond Detection of Physiological Adenosine Concentrations Using Fast-Scan Cyclic Voltammetry, Anal. Chem., 2007, 79 (2), pp 744-750

In summary, significant progress has been made in developing systems and methods for in-vivo chemical sensing of neurotransmitters and neuroactive substances. However, there remains a need for improved or alternative micro-electrode sensors and methods of fabrication of microelectrode sensors and sensor arrays for in vivo chemical sensing which address one or more of the above mentioned problems or disadvantages.

### SUMMARY OF INVENTION

The present invention seeks to provide a micro-electrode sensor and sensor arrays for chemical sensing that address one of more limitations or disadvantages of known microelectrode sensors for in vivo sensing, e.g. for monitoring of neurotransmitters and neuroactive substances.

Thus aspects of the invention provide conductive nanocrystalline diamond microelectrode sensors, micro-electrode sensor arrays and methods of fabrication thereof.

One aspect of the invention provides a micro-electrode sensor for in-vivo chemical sensing, comprising:

a conductive microwire having a distal end portion comprising a tip,

a layer of nanocrystalline conductive diamond deposited on at least the distal end portion of the conductive microwire,

an overlying layer of a biocompatible insulating material extending over the conductive microwire and part of the layer of conductive diamond, and one or more sensor areas of the conductive layer of diamond, each sensor area defined by a respective opening in the insulating material exposing a surface of the conductive diamond layer.

For example, the conductive microwire comprises a metallic microwire, e.g. of diameter of 150 μm or less, such as a tungsten, tantalum, molybdenum, or niobium microwire. The layer of conductive diamond preferably comprises boron-doped diamond. The diamond surface preferably has a surface roughness substantially less than 500 nm rms. Advantageously, the conductive diamond layer comprises nanocrystalline diamond or ultrananocrystalline diamond. For example the diamond layer may have a rms surface roughness and average grain size of ˜20 nm, and preferably ˜10 nm, or less. The layer of insulating material is preferably a good insulator that may be 5 μm or less thick, and comprises a biocompatible material, such as aluminum oxide, glass, or a biocompatible polymer such as parylene, or alternatively, a layer of non-conductive diamond, preferably non-conductive nanocrystalline diamond or ultrananocrystalline diamond (UNCD).

The one or more sensor areas of the conductive diamond layer may be surface treated to chemically modify the conductive diamond surface, or the sensor areas may further comprise a coating, e.g. of a neuroactive substance, to improve chemical and electrical sensitivity and selectivity.

Since a sensor area or a plurality of sensor areas are defined by openings in the insulating layer, the one or more sensor areas may be arranged on the distal portion of the micro-electrode sensor to sense only particular areas of interest in vivo. That is the sensor area or areas may be limited to reduce interference from surrounding areas, thus increasing the signal to noise ratio, and improving sensitivity and selectivity.

For example, in one embodiment the distal end portion of the microwire may have a blunt or cylindrical tip and an exposed cylindrical surface of the layer of conductive diamond forms a sensor area around the circumference of the microwire, while an end surface the microwire is uncoated by conductive diamond, i.e. the end of the metal microwire may be exposed or coated with another material, such as an insulating material.

In another embodiment, the distal end portion of the microwire has a blunt or cylindrical tip and both the cylindrical surface and the end surface of the microwire are coated with the layer of conductive diamond to form the sensor area.

In another embodiment the distal end portion of the microwire comprises a tapered portion, tapering to a narrow or sharp tip and the one or more sensor areas are provided along a length of the tapered portion including the sharp tip.

In another embodiment the distal end portion of the microwire comprises a tapered portion tapering to a narrow or sharp tip and the one or more sensor areas are provided along a length of the tapered portion spaced from the tip. The sharp tip is coated with insulating material.

One or more sensor areas may be defined by a plurality of openings in the insulating layer spaced apart along a length of the distal end portion, spaced from the tip.

When distal end portion comprises a tapered portion tapering to a narrow tip, the one or more sensor areas may be defined by a plurality of openings in the insulating layer spaced apart along a length of tapered portion spaced from the tip.

When the distal end portion comprises a tapered portion tapering to a narrow tip, the one or more sensor areas may be defined by an opening in the insulating layer exposing the narrow tip.

A plurality of sensor areas can be defined by openings in the insulating material defining a two dimensional pattern of sensor areas along the distal end portion. Alternatively, a plurality of sensor areas can defined by openings in the insulating material defining a three dimensional pattern of sensor areas over the surface of the distal end portion.

One or more sensor areas may be defined along a length of 500 μm or less of the distal end portion near the tip and distal end portion may comprise a tapered portion which tapers to a sharpened tip of about 1 μm in diameter. The one or more sensor areas may include a sensor area at the tip or be spaced from the tip, e.g. at least 10 μm from the tip.

Another aspect of the invention provides a micro-electrode array sensor comprising an assembly of an array of plurality of micro-electrode sensors, for example an array of a two dimensional pattern or three dimensional pattern of micro-electrodes. The array may comprise multiple discrete sensors or it may be an array of a plurality of sensors micro-patterned on a planar substrate. For example a plurality of microwires may be patterned on a common substrate, and then coated with a conductive diamond layer.

As an example, an array of a plurality of microelectrodes sensors may be configured as neurostimulation electrode sensors, i.e. having a “stimulation-recording-detection” capability.

Yet another aspect of the invention provides a method of fabricating a micro-electrode sensor for in-vivo chemical sensing comprising:

a) providing a conductive microwire comprising a distal end portion having a tip;

b) depositing a conductive diamond layer on at least the distal end portion of the conductive microwire;

c) depositing a biocompatible insulating layer over the conductive microwire and the conductive diamond layer;

d) selectively removing part of the insulating layer overlying the conductive diamond layer to expose one or more sensor areas of the conductive diamond layer.

The method may further comprise surface treating the exposed sensor area of the conductive diamond layer to chemically modify the surface of the exposed sensor areas, e.g. plasma cleaning or electrochemical cleaning of the exposed sensor area.

The step of selectively removing part of the insulating layer comprises etching using a chemical, electrochemical, and/or laser process to pattern and expose said one or more sensor areas. For example, the insulating layer may be selectively removed from a sensor area at the tip of the microwire and/or selectively removed from one or more sensor areas of the distal end portion spaced from the tip.

Alternatively the insulating layer may be selectively deposited to leave sensor areas of the conductive diamond layer exposed.

Optionally, the method further comprises modifying the one or more sensor areas of the exposed conductive diamond with oxygen-containing functional groups, enzymes and other bio layers for selective detection of neuro-active substances, non-electroactive chemicals and other electroactive chemicals. Examples include a neuro-active substance comprising hydrogen peroxide or oxygen or an electroactive chemical comprising adenosine.

For example, the method may further comprise modifying the one or more sensor areas of the exposed conductive diamond with oxygen-containing functional groups comprising at least one of hydroxyl, carbonyl, and carboxylic groups. Modification of the exposed conductive diamond may be made to enhance detection and focus the detection on more specific neuro-active substances, non-electroactive chemicals and other electroactive chemicals.

The diamond micro-electrode sensors and sensor arrays may be configured for fast-scan cyclic voltammetry (FSCV) for chemical monitoring.

The use of nanocrystalline diamond (NCD) or ultrananocrystalline diamond (UNCD) microsensors can provide higher sensitivity, faster response time and reduced fouling and tissue interaction. For FSCV, they offer improved temporal and chemical resolution and help to realize the full potential of FSCV. UNCD microelectrodes can match or exceed the key characteristics of carbon fiber microelectrodes in response time, spatial resolution, sensitivity, and the minimization of tissue disruption.

The sensors have high surface stability due to the extreme chemical inertness of UNCD/NCD, high reproducibility from the extremely low background charging current arising from an ultra-smooth surface and sp3 carbon microstructure, and high sensitivity associated with individually electrically addressable ultra-small electrode sizes.

A highly reliable electrode-electrolyte interface can be achieved by using a patterned, ultra-smooth conductive UNCD/NCD micro-electrode array. The chemical inertness of BDD electrodes enables them to be used as long term implantable microsensors.

Thus improved or alternative microelectrode sensors, micro-electrode sensor arrays and methods of fabrication of micro-electrode sensors. The conductive diamond micro-electrode sensors are preferably fabricated with UNCD sensor areas.

The foregoing and other objects, features, aspects and advantages of the present invention will become more apparent from the following detailed description, taken in conjunction with the accompanying drawings, of preferred embodiments of the invention, which description is by way of example only.

### BRIEF DESCRIPTION OF DRAWINGS

In the drawings, identical or corresponding elements in the different Figures have the same reference numeral.

FIG. 1 illustrates schematically a conventional carbon fiber microelectrode (CFM);

FIG. 2A illustrates schematically a micro-electrode sensor according to a first embodiment, in the form of a conductive microwire comprising a distal end portion having a conductive diamond sensor area at the tip;

FIG. 2B illustrates schematically an enlarged view of the distal end portion showing the exposed conductive diamond sensor area;

FIG. 3 illustrates schematically a cross-sectional view of the sensor of FIG. 2;

FIG. 4 shows a SEM image of a the tip of a micro-electrode sensor having a structure similar to that illustrated schematically in FIG. 3;

FIG. 5 illustrates schematically a micro-electrode sensor according to a second embodiment wherein the end of the sensor tip comprises UNCD;

FIG. 6 illustrates schematically a micro-electrode sensor according to a third embodiment comprising a tapered distal end portion, wherein the sensor area comprises a sharpened tip coated with UNCD;

FIG. 7 illustrates schematically a micro-electrode sensor according to a fourth embodiment wherein a plurality of UNCD sensor areas are defined by openings in the insulating layer along a length of a sharpened tip;

FIG. 8 illustrates schematically a micro-electrode sensor according to a fifth embodiment wherein a plurality of UNCD sensor areas are defined by openings in the insulating layer along a length of the tapered portion near the tip and wherein the tip is coated with insulating material;

FIG. 9 illustrates schematically a micro-electrode sensor according to yet another embodiment wherein one sensor area comprises a sharpened tip coated with UNCD and a plurality of sensor areas are defined by openings in the insulating layer spaced apart along a length of the distal end portion near the tip;

FIG. 10 shows a voltammogram comparing results for detection of dopamine using an untreated 200 um microdisk diamond surface and a UV treated 200 um microdisk diamond surface;

FIG. 11 shows a background cyclic voltammogram comparing results obtained with a CFM and a UNCD microelectrode for measuring dopamine;

FIG. 12 shows a voltammogram comparing results for a CFM and a UNCD microelectrode in response to a 10 second, 1 μM injection of dopamine; and

FIG. 13 shows a voltammogram comparing results for a CFM and a UNCD microelectrode in response to a 10 second, 1 μM injection of dopamine after subtracting the background signal.

### DETAILED DESCRIPTION OF EMBODIMENTS

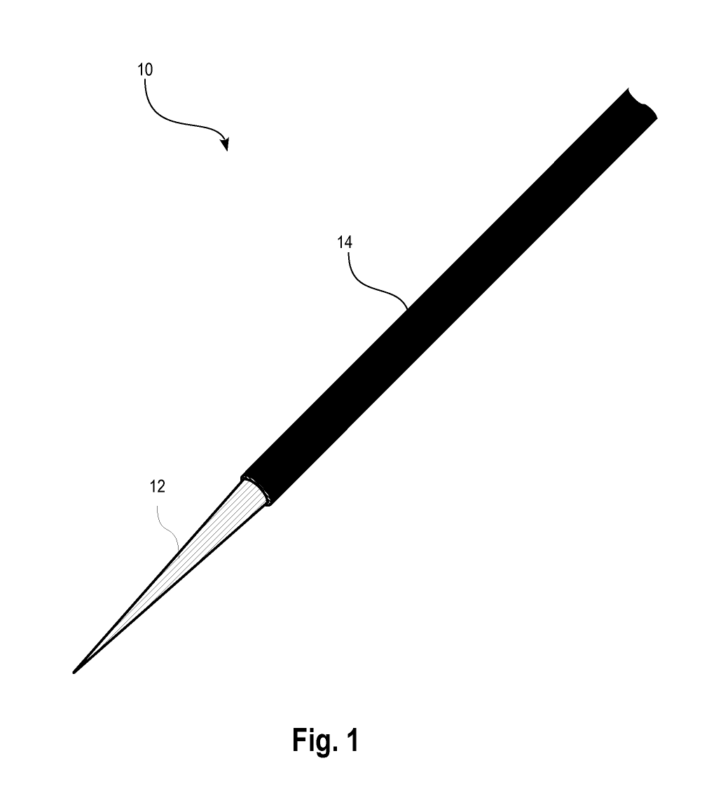

FIG. 1 illustrates schematically a conventional (prior art) carbon fiber microelectrode (CFM) 10 which comprises a carbon fiber 12, of e.g. 10 μm diameter and a glass insulating layer 14. The carbon fiber 12 typically may be ground or tapered to a narrow point at its distal end. The carbon fiber 12 is in electrical contact with a conventional conductor at the proximal end.

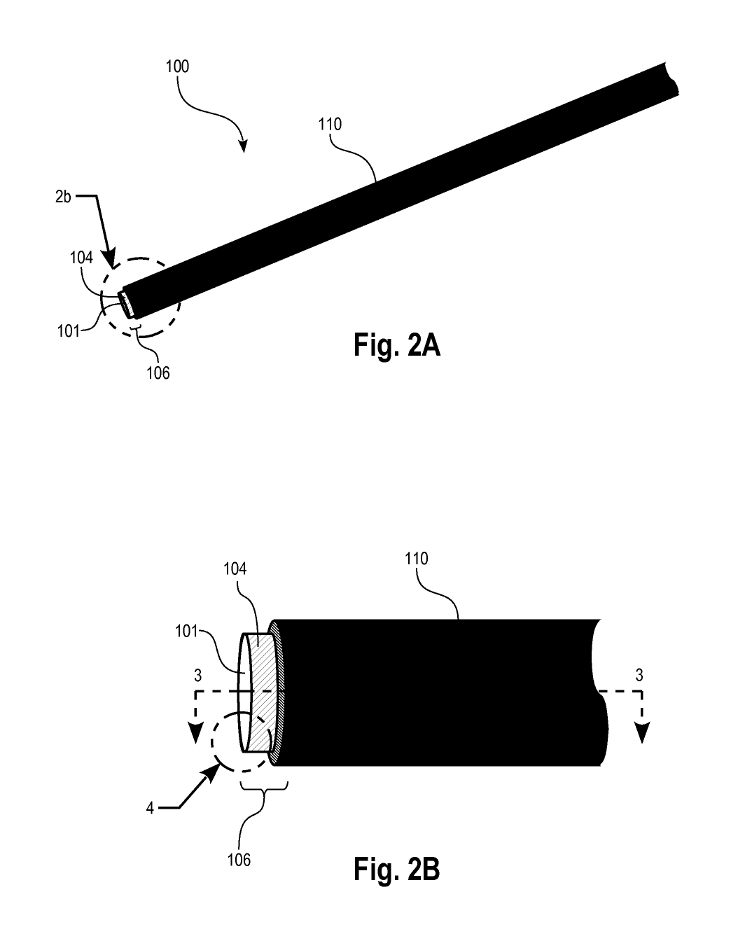

FIG. 2A illustrates schematically a micro-electrode sensor 100 or “probe” according to a first embodiment, suitable for in vivo sensing of neurotransmitters. The sensor 100 comprises a conductive microwire 101, e.g. a metallic microwire of tungsten or other suitable metal, having a distal end portion which comprises a coating of conductive diamond, such as boron doped diamond (BDD), 104, defining sensor area 106 at the tip. The microwire is coated with a biocompatible insulating material 110, such as aluminum oxide or parylene, that has minimal reaction to the surrounding biomaterial in which it is implanted.

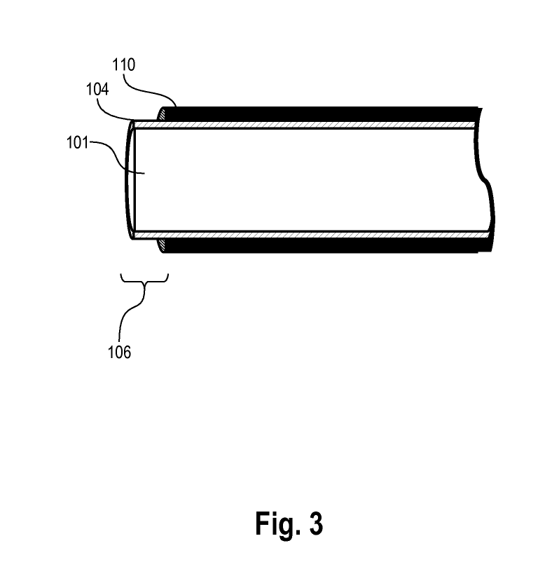

FIG. 2B illustrates schematically an enlarged view of the distal end portion showing the exposed conductive diamond sensor area 106. FIG. 3 illustrates a cross-sectional view of the sensor of FIGS. 2A and 2B. Preferably the conductive diamond layer is UNCD, which provides a sensor area having a very smooth surface, e.g. <10 nm rms surface roughness. As an example, the microwire may be fabricated having a length of 500 μm, a diameter of 30 μm and the insulating layer may be about 5 μm thick.

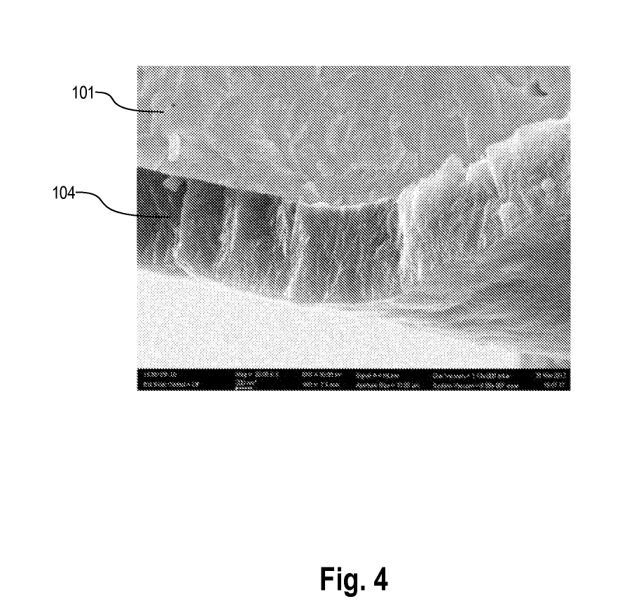

FIG. 4 shows a SEM image of the tip of a micro-electrode sensor of a structure similar to that illustrated schematically in FIGS. 2 and 3, comprising a tungsten microwire coated with conductive boron doped UNCD. As an example, the sensor shown in FIG. 4 comprises a microwire having a length of 1 to 3 mm and a diameter of less than 150 μm, the UNCD layer has a thickness of about 30 to 3000 nm and the insulating layer is about 5 μm thick.

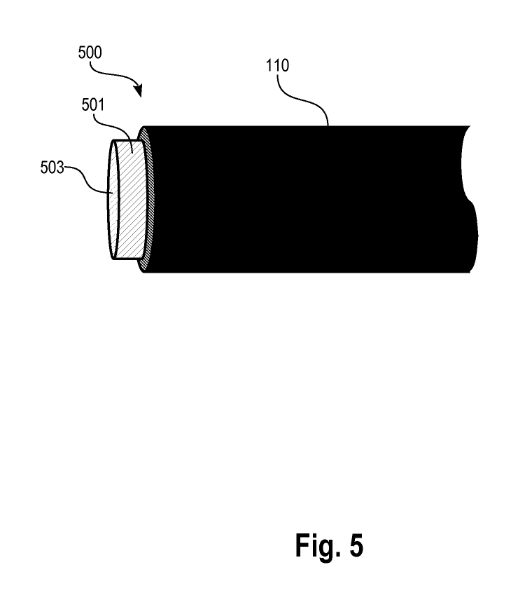

FIG. 5 illustrates schematically a micro-electrode sensor 500 according to a second embodiment, similar to that shown in FIGS. 2 and 3, but in which the end of the sensor tip 503 is coated with a layer conductive diamond which extends circumferentially around the cylindrical surface 501 beyond the insulating layer 110 and over the end 503 of the tip. The end coating 503 may provide further protection to the conductive microwire and in use, reduces the reactions of the microwire with the surrounding tissue.

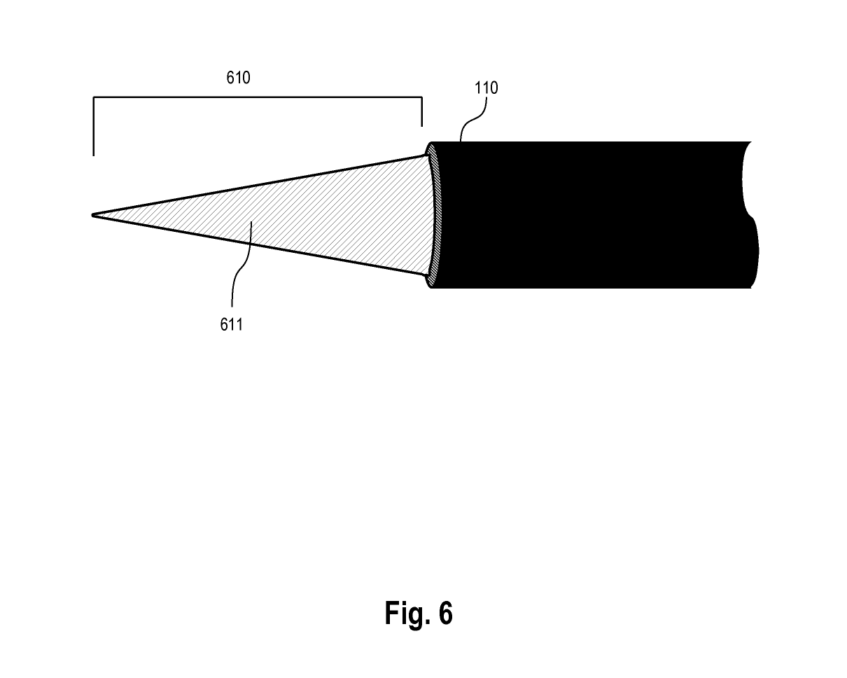

FIG. 6 illustrates schematically a micro-electrode sensor 600 according to a third embodiment comprising a tapered distal end portion 610 beyond the insulating layer 110, wherein the sensor area comprises a narrow or sharpened tip coated with UNCD. In some preferred embodiments, the microwire is sharpened to have a final tip point diameter of less than 2 μm or less than 1 μm. In use, tapering or sharpening the microwire to a fine point may reduce the interference of blood flow through capillaries in the test area. There may also be reduced damage to tissue and less interference to neuronal functions such as neurotransmitter release which is often caused by larger objects irritating the tissue.

The tip microwire is shaped or tapered, by conventional prior art etching or lapping, and then coated with the conductive diamond layer 611. For a simple structure where the entire tip forms a sensor area, the insulating material 110 may be selectively deposited on the microwire to leave the diamond tip exposed.

The sensing area of tip at the distal end may comprise one or more diamond sensing areas over a length of e.g. approximately 500 μm or less, or a length of 250 μm or less, and may include the tip or be spaced from the tip.

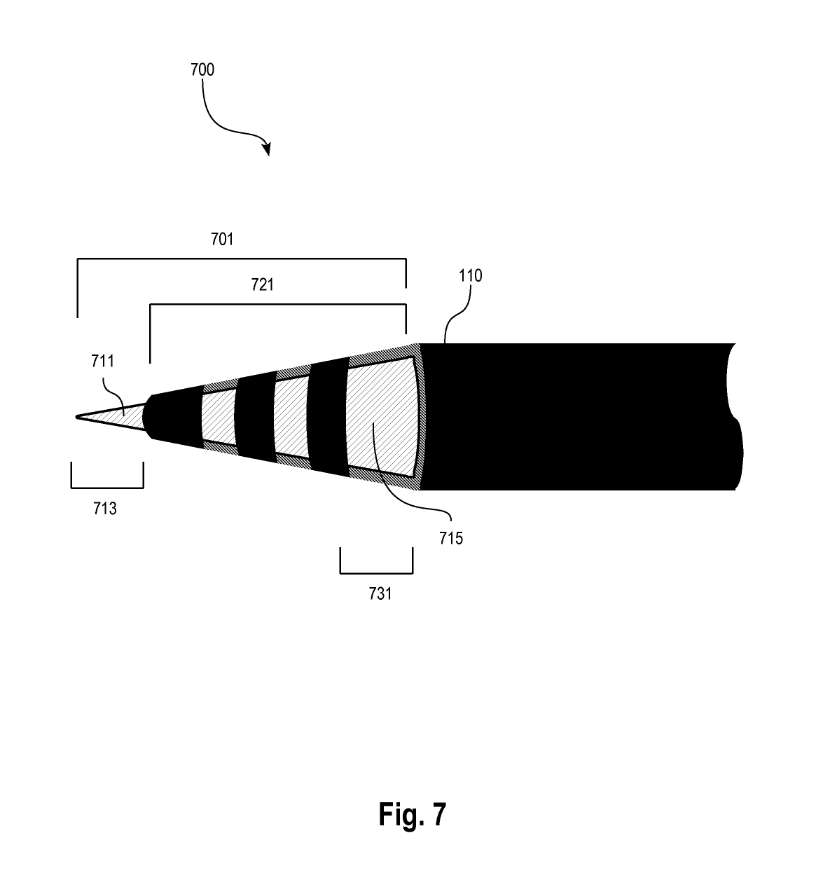

FIG. 7 illustrates schematically a micro-electrode sensor 700 according to a fourth embodiment wherein the sensor has a tapered distal portion 701. The insulating layer also extends over parts of the tapered portion. A plurality of UNCD sensor areas are defined by circumferential openings 731 in the insulating layer 110 along a length 721 of a sharpened tip, providing sensor areas 715. The sharpened tip 713 itself also provides a sensor area 711.

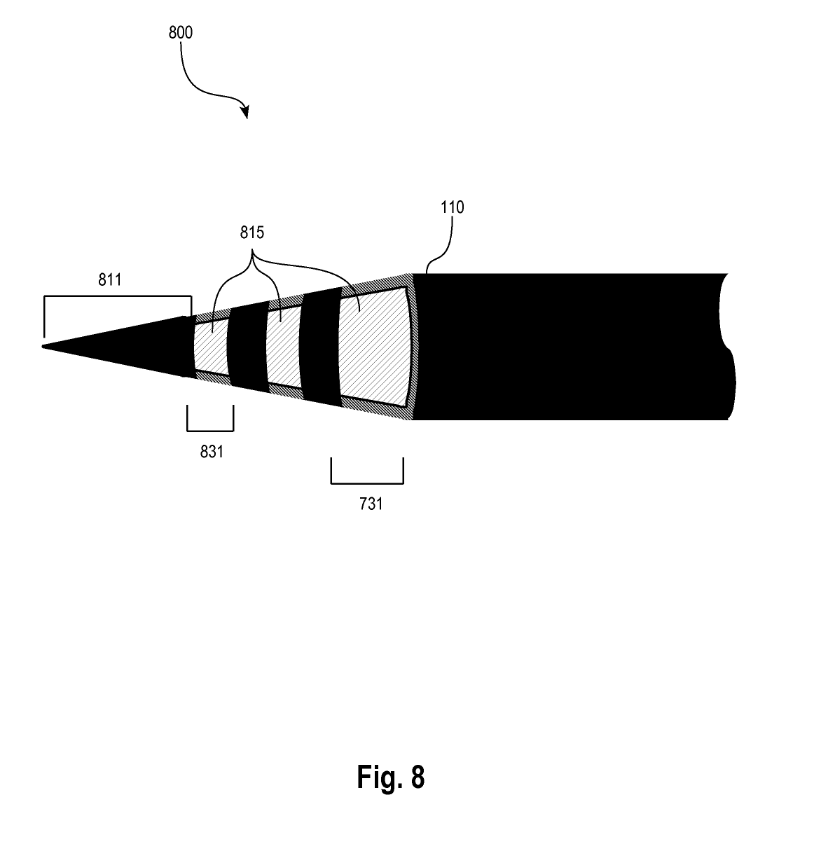

FIG. 8 illustrates schematically a micro-electrode sensor 800 according to a fifth embodiment, similar to that shown in FIG. 7, wherein a plurality of UNCD sensor areas 815 are defined by openings in the insulating layer 110 along a length of the tapered portion near the tip. In this embodiment the tip 811 is also coated with insulating material 110. An exposed diamond coated metal tip may be very fragile. Thus after coating the entire tip with insulating material 110, the insulating material 110 is selectively removed, e.g. by selective etching to opens up openings or windows 731 to form diamond sensing areas 815, leaving the tip 811 coated with insulating material. Leaving the insulating material at the very end of the tip provides additional strength, reducing the potential of tip damage. In some embodiments, this insulating material may extend at least 10 μm from the apex of the tip before the first window 831. By way of example, three windows are shown on the sharpened tip. However, it will be apparent that other numbers of windows may be provided to define one or more diamond sensing areas on the tapered portion and/or on the untapered portion of the distal end of the microwire.

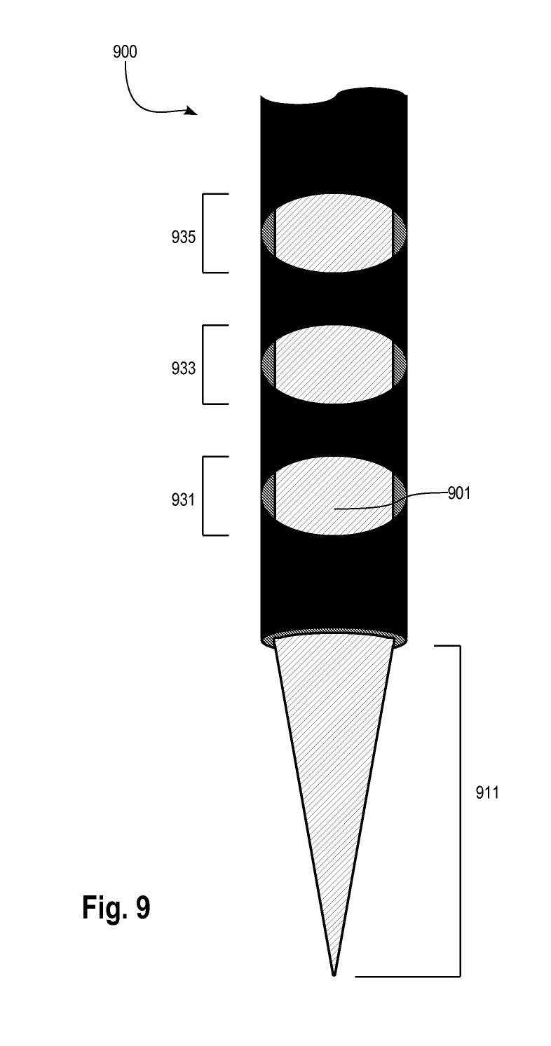

Thus, FIG. 9 illustrates schematically a micro-electrode sensor 900 according to yet another embodiment wherein one sensor area comprises a sharpened tip 911 coated with UNCD and a plurality of sensor areas 901 are defined by openings 931, 933, 931 in the insulating layer, spaced apart along a length of the distal end portion near the tip;

In the embodiments described above, the microwire runs through the center of at least the distal portion of the microelectrode sensor. In some embodiments, this microwire may run the full length of the microelectrode sensor. In other embodiments the microwire runs only through the distal portion and the microwire is electrically connected to a conventional larger diameter (non-microwire) conductor for electrical connection at the proximal end.

Suitable materials for the microwire include tungsten (W), tantalum (Ta), niobium (Nb), or molybdenum (Mo) which provide an appropriate substrate on which nanocrystalline or ultrananocrystalline diamond may be deposited. Other conductive materials such as titanium (Ti), silicon (Si) or even possibly carbon fibers, may be used as the substrate material for the microwire on which the conductive NCD or UNCD layer is deposited. In some embodiments, a conductive adhesion layer, e.g. comprising titanium nitride, or the metals and materials listed above may be deposited on the microwire prior to the deposition of the conductive diamond to enhance the adhesion of the diamond to the microwire.

The microwire is sized for strength and flexibility while maintaining a small enough diameter that will minimize damage to the organ in which it is inserted. For example, the diameter of the microwire may be in a range of 25 μm to 300 μm. For some applications, diameter may preferably be 150 μm or less. Typically the length of the microwire may be about one to three millimeters in length.

Beneficially, the conductive diamond layer comprises nanocrystalline diamond (NCD) or more preferably ultrananocrystalline diamond (UNCD). The small grain size of the deposited diamond results in a significant reduction in interaction with the surrounding tissue compared to other diamond deposition grain sizes and carbon fiber microelectrodes (CFM). Not only does the smaller grain size reduce the occurrence of pin holes but causes less tissue damage, and reduced surface fouling and surface adsorption of biomaterials. For example, UNCD has a near atomic scale roughness of ˜5 to 8 nm rms. This is significantly less that the surface roughness of 500-1000 nm rms of conventional microcrystalline diamond (MCD) based electrodes which are currently standard for in vivo measurements.

UNCD further provides other excellent performance characteristics under much more extreme conditions than those typically encountered for in vivo applications. These characteristics include low background currents, dimensional stability at high current densities and potentials (e.g., 1 A/cm² or greater for 100 hours at ˜7V wide electrochemical over-potentials for monitoring O₂ and H₂ evolution, and long electrode working lifetimes with a high level of physical and chemical inertness. For in vivo applications, it is advantageous to have low background current, i.e. to increase signal to noise ratios. Long lifetime with resistance to chemical or physical degradation or fouling is also beneficial for implantable micro-electrodes to be used for longer term in vivo sensing.

Using UNCD micro-electrode sensors as described above for in vivo sensing by electroanalytical methods known by those skilled in the art, detection limits beyond those used by other current state of the art in vivo sensors, such as CFM, can be realized. For sensors according to some embodiments, for example, it has been demonstrated that dopamine can be detected at levels less than 100 nM. In exemplary experiments, the detected level of dopamine is less than 10 nM. It was observed that the UNCD conductive diamond sensing area provided faster response times, e.g. less than 200 ms, relative to CFM.

Furthermore, microelectrode sensors according to embodiments of the invention are not limited to neurotransmitter sensing. Other analytes and conditions may be sensed, such as changes in pH or ferrocyanide/ferricyanide concentrations. If required, the diamond surface of the sensor areas may be modified to improve sensitivity and selectivity, e.g. by hydrogen or oxygen treatment or by functionalization of the surface with active species for the detection of certain chemicals or even for biosensing

Fabrication.

Micro-electrode sensors as described above may be fabricated by method steps comprising: providing a conductive microwire comprising a distal end portion having a tip; depositing a conductive diamond layer on at least the distal end portion of the conductive microwire; depositing a biocompatible insulating layer over the conductive microwire and the conductive diamond layer; and selectively removing part of the insulating layer overlying the conductive diamond layer to expose one or more sensor areas of the conductive diamond layer.

Once the surface of the microwire is prepared for diamond deposition, e.g. by steps of roughening (lapping or bead blasting) or chemical etching, a layer of conductive diamond is deposited thereon. For example, the diamond layer may comprise boron doped UNCD deposited by Hot Filament Chemical Vapour deposition from a reactant gas mixture comprising methane and hydrogen (CH₄/H₂) with a boron dopant gas, such as trimethyl borane.

The layer of conductive diamond may be deposited to a thickness in the range from 50 nm to 3000 nm, although for some embodiments the diamond layer may be deposited to a thickness greater than 1500 nm or 1.5 μm.

The NCD/UNCD diamond deposition process is optimized to avoid pin holes and reduce stress and graphite deposition. Pin holes through the diamond to certain substrates metals such as tungsten or titanium may produce undesirable signals or variations or cause deterioration to the substrate. Pin holes expose the metal underneath the diamond coatings which increases the background charging current and affects sensitivity. Optimization of grain size and diamond layer or film thickness can reduce the occurrence of these pin holes.

Graphite can also cause interference to readings due to its inferior electrochemical properties compared to the conductive diamond. Increased graphite deposition is often a result of non-uniform temperatures in the local deposition area or catalytic activity as a result of other materials exposed in the deposition process. Adjustments to the reactor configuration may help in providing more uniform temperatures. Also, operation at higher temperatures (such as >730° C.) and longer processing times (>25 minutes) may further provide for temperature uniformity.

The diamond film 104 stress will directly affect the adhesion of the diamond to the substrate 101. For deposition or NCD and UNCD by HFCVD, the CH₄/H₂ ratio can be reduced to generate more atomic hydrogen which enhances filament decarburization, raises substrate temperatures and reduces stress in the as-deposited film

If required, the diamond surface may be modified once the diamond film is deposited. For example, a post treatment with atomic hydrogen or oxygen may be used, e.g. to improve the selectively of the diamond surface to neuroactive analytes.

An insulating layer comprising a suitable biocompatible material is then formed onto the microwire and overlying the diamond film. The insulating layer leaves one or more areas of the conductive diamond layer exposed to define each sensing area. There are many biocompatible insulating materials that may be used for this insulating material. These include aluminum oxide, a polymer such as parylene, glass and a non-conductive diamond layer. Beneficially, the insulating layer is a good insulator that may be deposited to a thickness of 5 μm or less.

For simple structures with one sensing area at the tip, the insulating material may be selectively deposited on the diamond coated wire in a manner to leave the tip exposed. In other embodiments, surfaces of the microwire may be coated with the insulating material and then the insulating material is selectively removed to form openings defining sensor areas.

There are many methods that may be employed to selectively remove the insulation to form openings defining sensing areas of the conductive diamond surface. The methods may be selected depending on the insulating material used. Removal methods may include mechanical abrasion, chemical etching (i.e. wet etching of an oxide layer) and laser etching. Most of the material may be removed with an etching process but in some embodiments, a further cleaning process may be needed to remove any residual insulating material. Such processes may include electrochemical cleaning or a more rigorous oxygen plasma cleaning process. The latter cleaning process is preferably applied when it is desired to improve the attainable signal-to-noise ratio (S/N) to at least 25.

In some embodiments, e.g as shown in FIG. 7, instead of exposing a substantial portion of the tip 610, smaller portions 731 are exposed. For a tapered or sharpened tip, the microwire is first tapered or shaped before coating with conductive diamond. After applying insulating material 110 over the tip and other parts of the microwire, selective etching is used to expose portions 711, 715 of the tip 701 defining the sensing areas. In some embodiments, the very tip of the insulated microwire 700 may be exposed to form an exposed tip 713. In some embodiments, this exposed tip 713 may be less than 50 μm and in further embodiments, less than 25 μm. Additional windows 731 may be etched through the insulating material 721. This will expose further sensing areas and thus providing better sensitivity in a specified location and direction. Effectively, this process provides a patterned electrode over the distal portion of the microwire.

In variants of these embodiments, the tip may be a blunt or cylindrical tip (examples shown in FIGS. 2 and 5) or as a tapered or sharp pointed tip (examples in FIGS. 6 and 7). Further, one or more windows can be selectively etched into the unsharpened or untapered portion of the electrode and/or in the tapered portion.

Surface Treatments

In some embodiments, it may be beneficial if the conductive diamond is further surface treated or additional layers may be deposited. Surface treatment may be done prior to applying the insulating layer or surface treatment of the exposed sensing areas or it may be done after the selective removal of the insulating layer to open windows defining the sensing areas. The surface treatment may be a surface modification or deposition of a surface layer, e.g. enzymes or other bio layers. This additional treatment may be provided to improve selective detection of non-electroactive chemicals.

Micro-Electrode Sensor Arrays

In another embodiment, several electrodes in an in vivo sensor array may be used to provide spatial sensing, e.g. for an area of the brain tissue. A common configuration is a 4×4 multi micro-electrode array. With the use of FSCV and high speed multiplexing methods, individual data can be collected for each of the electrodes and then analyzed, producing a 3-D spatial sensing of the tissue area.

Individual probes may be combined to produce the array or, in alternative embodiments, a plurality of electrodes may be patterned on a single substrate. This substrate may include the microwires.

In other embodiments of a micro-electrode array, the array may further comprise some neurostimulation electrodes. These electrodes may be used for stimulating the local brain tissue to provide a response for the detection of neurochemical/neurotransmitter sensors. This provides a “stimulation-recording-detection” capability to the array. With the combination of stimulation and sensing or recording electrodes in an array, this array may be part of a universal platform for many applications requiring stimulation, recording, and sensing functions.

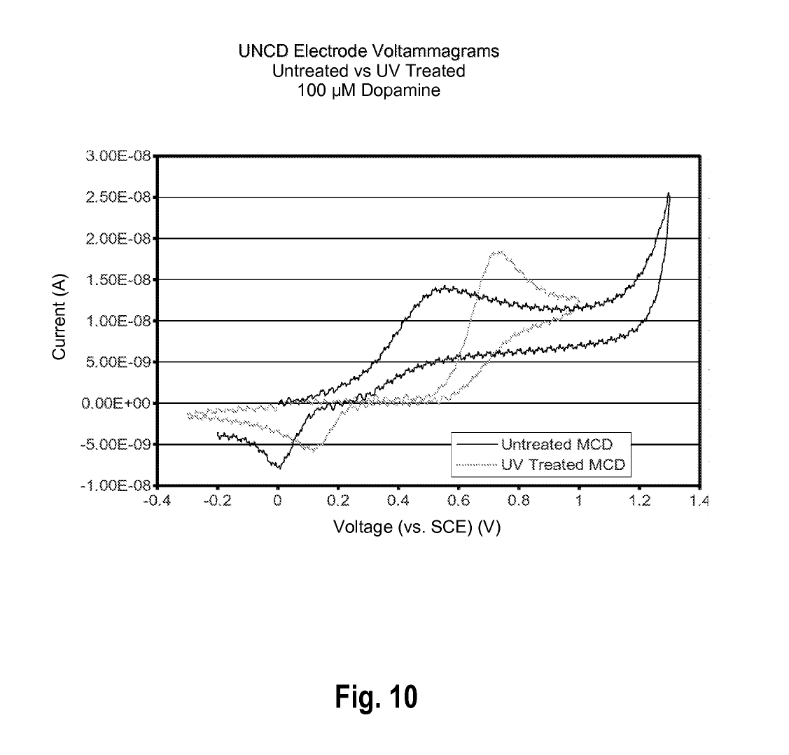

In some embodiments, the exposed conductive diamond surfaces forming the sensor areas may be treated with ultraviolet light (UV) to enhance the detection of the neurotransmitters. FIG. 10 shows experimental results comparing untreated and UV treated 200 μm microdisks having conductive diamond surfaces. For UV treatment, the surface was exposed to 254 nm UV light for 60 minutes and then used to detect 100 μM dopamine in a solution. UV treatment introduced hydroxyl groups on the surface, which renders the surface more hydrophilic and thus improves dopamine adsorption. UV treatment primarily reduces surface sp² bonds and introduces oxygen-containing surface groups. As illustrated by the experimental results, UV treatment significantly increased the sensitivity of dopamine signal by 45%. That is, the oxidation peak current, which is the dopamine signal, increased from 8.3 to 12.3 nA and a substantial decrease in the background current was noted.

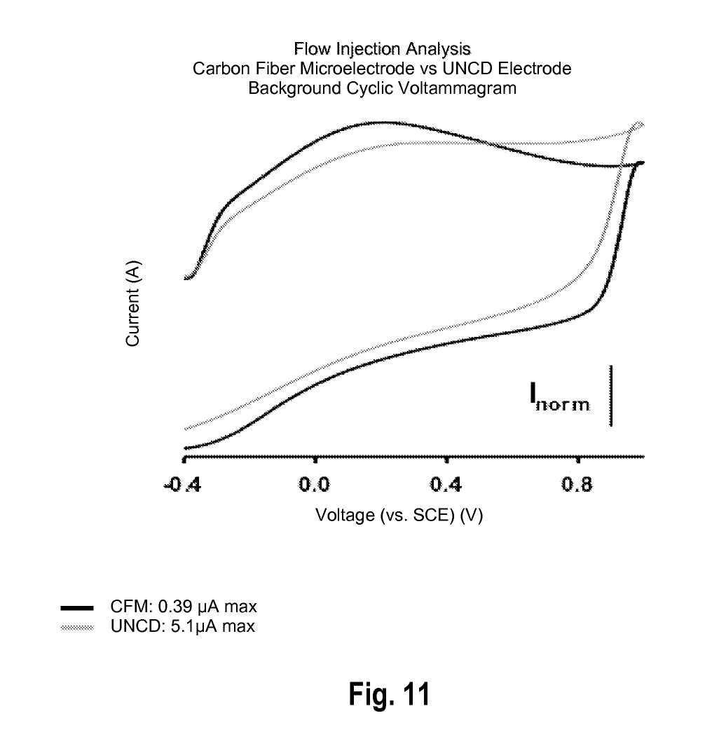

FIGS. 11, 12 and 13 compare experimental results obtained with a UNCD electrode (black curves) and a CFM electrode (gray curves) for measuring dopamine with flow injection analysis. The measurements were collected in buffered physiological saline. The reference electrode was a chloridized silver wire (Ag/AgCl). For FSCV, a triangle wave from +0.4 to +1.0 V and back was applied at a rate 300 V/s every 100 ms. A dopamine bolus of 1 or 10 μM was injected at 0 s for 10 s. The anodic current is positive. Current was monitored across the peak oxidative potential for dopamine (+0.6 V). Buffer flow rate was 3 ml/min FIG. 11 shows the FSCV measurement of the background prior to the addition of the dopamine. The UNCD had a max current of 5.1 μA while the CFM electrode had a max current of 0.39 μA.

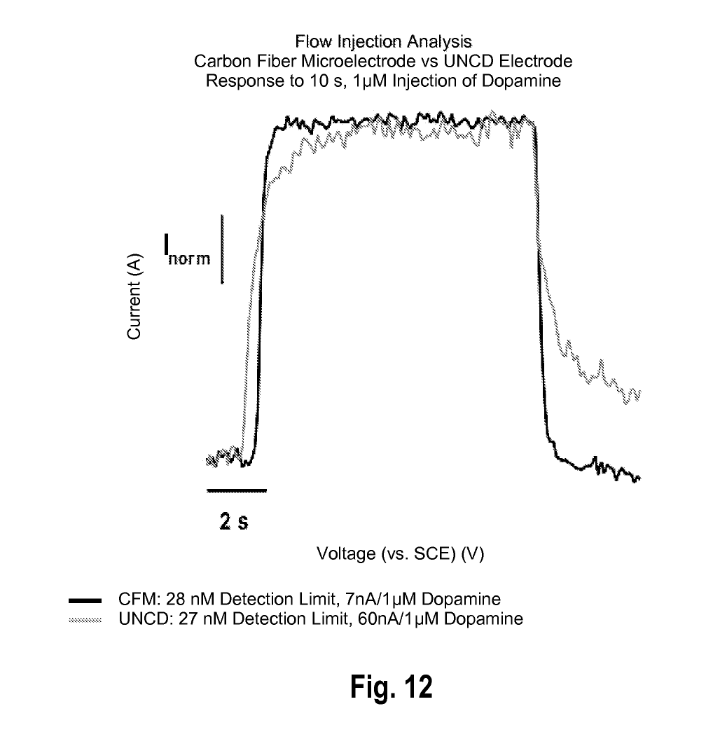

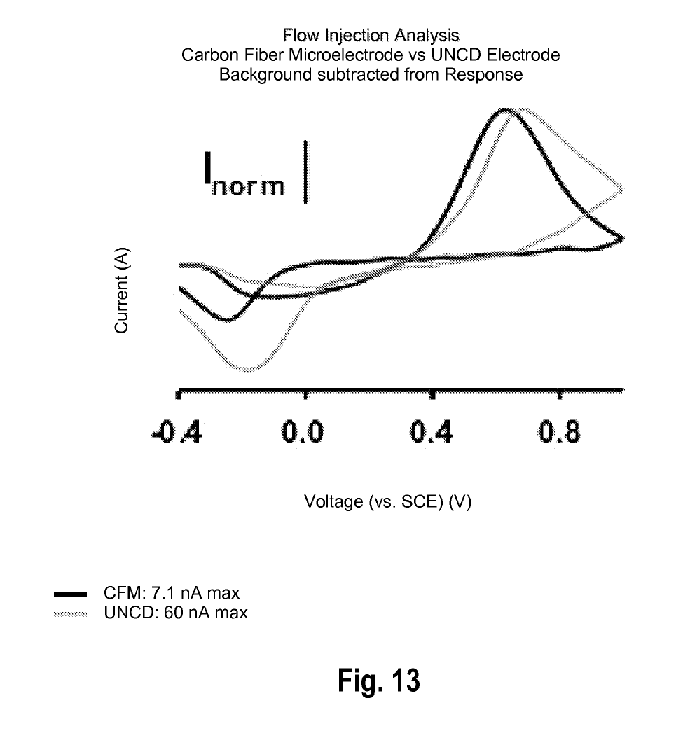

FIG. 12 shows the FSCV response to a 10 second, 1 μM injection of dopamine. The UNCD electrode had a detection limit of 27 nM and a sensitivity of 60 nA/1 μM while the CFM electrode had a detection limit of 28 nM and a sensitivity of 7 nA/1 μM. FIG. 13 shows the background subtracted from the response CV for the dopamine injection, with a maximum current of 60 nA and 7.1 nA for UNCD and CFM respectively.

### INDUSTRIAL APPLICABILITY

Conductive diamond micro-electrode sensors and sensor arrays according to embodiments of the present invention are provided which are suitable for in vivo chemical sensing. Also provided is a method of fabrication of individual micro-electrode sensors and sensor arrays. Reliable, sensitive and selective chemical micro-sensors may be constructed for real-time, continuous monitoring of neurotransmitters and neuro-active substances in vivo. In preferred embodiments, each sensor comprises conductive microwire, having a distal end comprising a tip, coated with nanocrystalline or ultrananocrystalline conductive diamond, and an overlying insulating layer. Active sensor areas of the conductive diamond layer are defined by openings in the insulating layer at the distal end. Multiple sensor areas may be defined by a 2 or 3 dimensional pattern of openings near the tip. Particular arrangements of defined conductive diamond sensor areas limits interference from surrounding areas for improved signal to noise ratio, sensitivity and selectivity. For example, for applications such as fast-scan cyclic voltammetry, multiple sensors can be arrayed and operated using high speed multiplexers, to provide 3-D spatial sensing with near real-time monitoring.

Although embodiments of the invention have been described and illustrated in detail, it is to be clearly understood that the same is by way of illustration and example only and not to be taken by way of limitation, the scope of the present invention being limited only by the appended claims.

1. A micro-electrode sensor for in-vivo chemical sensing, comprising:
a conductive microwire having a distal end portion comprising a tip,
a layer of nanocrystalline or ultrananocrystalline conductive diamond deposited on at least the distal end portion of the conductive microwire,
an overlying layer of a biocompatible insulating material extending over the conductive microwire and part of the layer of conductive diamond, and one or more sensor areas of the conductive layer of diamond on the distal end portion, each sensor area defined by a respective opening in the insulating material exposing a surface of the conductive diamond layer.

2. The sensor of claim 1, wherein the conductive microwire comprises a microwire of one or more of tungsten, tantalum, molybdenum, titanium and niobium.

3. The sensor of claim 1, wherein the layer of conductive diamond comprises boron-doped diamond.

4. The sensor of claim 1 wherein the conductive diamond has a roughness of substantially less than 500 nm rms.

5. The sensor of claim 1, wherein the conductive layer of diamond comprises nanocrystalline diamond or ultrananocrystalline diamond having a surface roughness of 20 nm rms or less.

6. The sensor of claim 5 wherein the thickness of the conductive diamond layer is from 30 nm to 3000 nm.

7. The sensor of claim 1, wherein the one or more sensor areas are surface treated to chemically modify the surface of the conductive diamond layer.

8. The sensor of claim 1 wherein the one or more sensor areas further comprises coating of a chemical sensor layer.

9. The sensor of claim 1 wherein the distal end portion of the microwire has a blunt tip and an exposed cylindrical surface of the layer of conductive diamond forms a sensor area and an end surface the microwire is uncoated.

10. The sensor of claim 1 wherein the distal end portion of the microwire has a blunt tip and a cylindrical surface and end surface of the blunt tip of the microwire is coated with the layer of conductive diamond to form the sensor area.

11. The sensor of claim 1 wherein the distal end portion of the microwire comprises a tapered portion tapering to a sharp tip and the one or more sensor areas are provided along a length of the tapered portion including the sharp tip.

12. The sensor of claim 1 wherein the distal end portion of the microwire comprises a tapered portion tapering to a sharp tip and the one or more sensor areas are provided along a length of the tapered portion spaced from the sharp tip.

13. The sensor of claim 11 wherein the sharp tip is coated with insulating material.

14. The sensor of claim 1 wherein the one or more sensor areas are defined by a plurality of openings in the insulating layer spaced apart along a length of the distal end portion spaced from the tip.

15. The sensor of claim 1 wherein the distal end portion comprises a tapered portion tapering to a narrow tip, the one or more sensor areas are defined by a plurality of openings in the insulating layer spaced apart along a length of tapered portion spaced from the tip.

16. The sensor of claim 1 wherein the distal end portion comprises a tapered portion tapering to a narrow tip, and a sensor area is defined by an opening in the insulating layer exposing the narrow tip.

17. The sensor of claim 1 wherein a plurality of sensor areas are defined by openings in the insulating material defining a two dimensional pattern of sensor areas along the distal end portion.

18. The sensor of claim 1 wherein a plurality of sensor areas are defined by openings in the insulating material defining a three dimensional pattern of sensor areas over the surface of the distal end portion.

19. The sensor of claim 1 wherein the diameter of the microwire is 150 μm or less.

20. The sensor of claim 1 wherein sensor areas are defined along a length of 500 μm or less of the distal end portion near the tip.

21. The sensor of claim 1 wherein the conductive wire has a diameter of 150 μm and the distal end portion tapers to a sharpened tip of about 1 μm in diameter.

22. The sensor of claim 18 wherein the one or more sensor areas comprise the sharpened tip along a length of 500 μm or less of the distal end portion adjacent the tip.

23. The sensor of claim 1 wherein the layer of insulating material is less than 5 μm thick.

24. The sensor of claim 1 wherein the one or more sensor areas are spaced at least 10 μm from the tip.

25. The sensor of claim 1 for use in fast scan cyclic voltammetry (FSCV), capable of providing a signal-to-noise ratio of at least 25.

26. The sensor of claim 1 for detection of dopamine at levels of less than 100 nM.

27. The sensor of claim 1 wherein the exposed conductive diamond surface is modified with oxygen-containing functional groups, enzymes and other bio layers for selective detection of neuro-active substances, non-electroactive chemicals and other electroactive chemicals.

28. The sensor of claim 27 wherein the oxygen-containing functional groups comprise at least one of hydroxyl, carbonyl, and carboxylic.

29. The sensor of claim 27 wherein the neuro-active substance comprises hydrogen peroxide or oxygen.

30. The sensor of claim 27 wherein the electroactive chemical is adenosine.

31. The micro-electrode array sensor comprising an assembly of an array of plurality of micro-electrode sensors as defined in claims 1 to 30.

32. The micro-electrode array sensor claim 31 wherein the microelectrodes sensors are configured as neurostimulation electrode sensors having a “stimulation-recording-detection” capability.

33. The sensor of claim 28 wherein the array comprises a two dimensional pattern of sensors.

34. The sensor of claim 28 wherein the array comprises a three dimensional pattern of sensors.

35. A method of fabricating a micro-electrode sensor for in-vivo chemical sensing comprising: a) providing a conductive microwire comprising a distal end portion having a tip;
b) depositing a conductive diamond layer on at least the distal end portion of the conductive microwire;
c) depositing a biocompatible insulating layer over the conductive microwire and the conductive diamond layer;
d) selectively removing part of the insulating layer overlying the conductive diamond layer to expose one or more sensor areas of the conductive diamond layer.

36. The method of claim 35 further comprising, surface treating the exposed sensor area of the conductive diamond layer to chemically modify the surface of the exposed sensor areas.

37. The method of claim 35 wherein surface treating comprises one or plasma cleaning or electrochemical cleaning of the exposed sensor area

38. The method of claim 29 wherein the step of selectively removing part of the insulating layer comprises etching using a chemical, electrochemical, and/or laser process to expose said one or more sensor areas.

39. The method of claim 35 comprising selectively removing the insulating material from a sensor area at the tip of the microwire.

40. The method of claim 35 comprising selectively removing the insulating material from one or more sensor areas of the distal end portion spaced from the tip.

41. The method of claim 35 wherein the tip of the microwire is tapered to a sharp tip, comprising selectively removing the insulating material from one or more sensor areas of the distal end portion leaving the tip coated with insulating material.

42. The method of claim 35 comprising selectively removing insulating material to form a plurality of opening in the insulating material along a length of the distal end portion near the tip.

43. The method of claim 35 wherein the distal end portion of microwire comprises a tapered portion which tapers to a sharp tip and selectively removing insulating material comprises forming a plurality of openings in the insulating material along the length of the tapered portion near the tip.

44. The method of claim 35 wherein the conductive microwire comprises a microwire of one of tungsten, tantalum, molybdenum, platinum, titanium and niobium, and wherein the step of depositing the conductive diamond layer comprises depositing conductive diamond layer comprises boron-doped diamond.

45. The method of claim 35 wherein the conductive microwire comprises a microwire of one of tungsten, tantalum, molybdenum, platinum, titanium and niobium method of claim 35 wherein the step of depositing the conductive diamond layer comprises depositing nanocrystalline diamond or ultrananocrystalline diamond.

46. The method of claim 35 wherein the step of depositing the biocompatible insulating material comprises depositing a layer of aluminum oxide, glass, parylene or non-conductive diamond.

47. The method of claim 35 further comprising modifying the one or more sensor areas of the exposed conductive diamond with oxygen-containing functional groups, enzymes and other bio layers for selective detection of neuro-active substances, non-electroactive chemicals and other electroactive chemicals.

48. The method of claim 35 further comprising modifying the one or more sensor areas of the exposed conductive diamond with oxygen-containing functional groups comprising at least one of hydroxyl, carbonyl, and carboxylic.

49. The method of claim 35 further comprising modifying the one or more sensor areas of the exposed conductive diamond with a neuro-active substance comprising hydrogen peroxide or oxygen.

50. The method of claim 35 further comprising modifying the one or more sensor areas of the exposed conductive diamond with an electroactive chemical comprising adenosine.
